(19) United States
(12) Patent Application Publication
(10) Pub. No.: US 2003/0040601 A1
(43) Pub. Date: Feb. 27, 2003

(54) Method for making insulin precursors and insulin analog precursors

(76) Inventors: Ivan Diers, Vaerlose (DK); Thomas Kjeldsen, Virum (DK)

Correspondence Address: NOVO NORDISK OF NORTH AMERICA, INC, 405 LEXINGTON AVENUE, SUITE 6400, NEW YORK, NY, 10017

(21) Appl. No.: 10/166,241

(22) Filed: Jun. 7, 2002

Related U.S. Application Data
(60) Provisional application No. 60/299,091, filed on Jun. 18, 2001.

(30) Foreign Application Priority Data
Jun. 8, 2001 (DK) PA 2001 00894

(51) Int. Cl. A61K 38/28; C07K 14/62

(52) U.S. Cl. 530/303; 514/3

(57) ABSTRACT

Novel insulin precursors and insulin analogue precursors comprising a connecting C-peptide and an N-terminal extension are easy to handle in down stream processing and are expressed in high yields. The precursors are characterized in that the connecting peptide, the N-terminal extension or both contain at least one glycosylation site.

### CROSS REFERENCE TO RELATED APPLICATIONS

This application claims priority under 35 U.S.C. 119 of Danish application no. PA 2001 00894 filed on Jun. 8, 2001, and U.S. provisional application No. 60/299,091 filed on Jun. 18, 2001, the contents of which are fully incorporated herein by reference.

### BACKGROUND

Yeast organisms produce a number of proteins that are transported through the secretory appatus (ER-Golgi-Secretory vesicles) and sorted to the medium or extracellular space. Such proteins are referred to as secreted proteins and they usually do a function outside the cell envelope. These proteins are initially expressed in the cytoplasm and cotranslationally translocated across the membrane of the endoplasmic reticulum (ER) in a precursor or a pre-form containing a pre-peptide sequence ensuring effective direction (translocation) of the expressed product across the membrane. The prepeptide, normally named a signal peptide, is generally cleaved off from the desired product during translocation. Small secreted proteins like the α-Mating Factor also contain a pro-region which is N-glycosylated providing proteolytic protection of the molecule, correct folding and transport and sorting. N-glycosylation takes place in the ER and in a cotranslational manner. Correctly folded molecules are further transported down the secretory pathway into the Golgi apparatus, where the core N-glycosylation is modified often leading to hyperglycosylated proteins. Finally proteolytic cleavage and modification can take place in a late Golgi compartment, as described for the α-Mating Factor, before the protein is sorted by different routes that lead to compartments such as the cell vacuole, or it can be routed out of the cell to be secreted to the external medium (Pfeffer et al. (1987) Ann. Rev. Biochem. 56:829-852).

Insulin is a polypeptide hormone secreted by β-cells of the pancreas and consists of two polypeptide chains, A and B, which are linked by two inter-chain disulphide bridges. Furthermore, the A-chain features one intra-chain disulphide bridge.

The hormone is synthesized as a single-chain precursor proinsulin (preproinsulin) consisting of a prepeptide of 24 amino acid followed by proinsulin containing 86 amino acids in the configuration: prepeptide -B-Arg Arg-C-Lys Arg-A, in which C is a connecting peptide of 31 amino acids. Arg-Arg and Lys-Arg are cleavage sites for cleavage of the connecting peptide from the A and B chains.

Three major methods have been used for the production of human insulin in microorganisms. Two involve Escherichia coli, with either the expression of a large fusion protein in the cytoplasm (Frank et al. (1981) in Peptides: Proceedings of the 7^{th} American Peptide Chemistry Symposium (Rich & Gross, eds.), Pierce Chemical Co., Rockford, Ill. pp 729-739), or use a signal peptide to enable secretion into the periplasmic space (Chan et al. (1981) PNAS 78:5401-5404). A third method utilizes Saccharomyces cerevisiae to secrete an insulin precursor into the medium (Thim et al. (1986) PNAS 83:6766-6770). The prior art discloses a limited number of insulin precursors which are expressed in either E. coli or Saccharomyces cerevisiae, vide U.S. Pat. No. 5,962,267, WO 95/16708, EP 0055945, EP 0163529, EP 0347845 and EP 0741188. The prior art further discloses expression of insulin precursors comprising certain N-terminal extensions and certain connecting peptides, vide WO 95/34666, WO 95/35384, WO 97/22706, EP 704527 and WO 98/28429.

The present invention provides for a method giving insulin precursors which are easy to handle in down stream purification steps such as centrifugation and filtration where it may be important that the product has a high solubility and a low tendency to form fibrils or a gel. The precursors will also be easy to separate by affinity chromatography. Finally, the precursors are expressed in high yields in transformed host cells.

### SUMMARY OF THE INVENTION

The present invention features novel insulin precursors and insulin analogue precursors comprising a connecting peptide (C-peptide) and an N-terminal extension wherein the connecting peptide or the N-terminal extension or both comprise at least one glycosylation site. Such insulin precursors or insulin analogue precursors can then be converted into human insulin or an insulin analogue by one or more suitable, well known conversion steps.

The connecting peptide will be of up to 10 or up to 5-8 amino acid residues or up to three amino acid residues in length.

The connecting peptide is to be cleavable from the A- and B-chains and will contain a cleavage site at its C-terminal end enabling in vitro cleavage of the connecting peptide from the A chain. Such cleavage site may be any convenient cleavage site known in the art, e.g. a Met cleavable by cyanogen bromide or an Asn, Asn-Gly cleavable with hydroxylamine; a single basic amino acid residue (Lys or Arg) cleavable by trypsin or trypsin like proteases; a lysine residue cleavable with Achromobacter lyticus protease or a pair of basic amino acid residues (Lys or Arg) cleavable by kexin or yapsin from yeast or their homologues from other eukaryotic organisms. The cleavage site enabling cleavage of the connecting peptide from the A-chain is preferably a single basic amino acid residue Lys or Arg, preferably Lys.

Cleavage of the connecting peptide from the B chain may conveniently be accomplished by cleavage at the natural Lys^{B29} amino acid residue in the B chain giving rise to a desB30 insulin precursor. If the insulin precursor is to be converted into human insulin, the B30 Thr amino acid residue (Thr) can then be added by well known in vitro, enzymatic procedures.

Cleavage from the B-chain may also be accomplished by insertion of a suitable cleavage site at the C-terminal end of the connecting peptide such as Met cleavable by cyanogen bromide or an Asn, Asn-Gly cleavable with hydroxylamine; a single basic amino acid residue (Lys or Arg) cleavable by trypsin or trypsin like proteases; a lysine residue cleavable with Achromobacter lyticus protease or a pair of basic amino acid residues (Lys or Arg) cleavable by kexin or yapsin from yeast or their homologues from other eukaryotic organisms.

In one embodiment the connecting peptide will not contain two adjacent basic amino acid residues (Lys,Arg). In this embodiment, cleavage from the A-chain may be accomplished at a single Lys or Arg located at the N-terminal end of the A-chain and the natural Lys in position B29 in the B-chain.

The insulin precursors or insulin analogue precursors according to the present invention will be expressed as a fusion protein comprising an N-terminal extension immediately N-terminal to the B-chain. The N-terminal extension will typically be of up to 30 amino acid residues in length and may contain at least one glycosylation site. The N-terminal extension will contain a cleavage site enabling its cleavage from the precursor molecule. Such cleavage site may by any convenient cleavage site well known in the art, such as Met or a mono or dibasic amino acid sequence Lys, Arg.

Thus, the present invention relates to insulin precursors or insulin analogue precursors comprising a connecting peptide (C-peptide) being cleavable from the A and B chains and an N-terminal extension immediately N-terminal to the N-terminal amino acid residue in the B-chain, wherein the connecting peptide, the N-terminal extension or both contain at least one glycosylation site and wherein the connecting peptide is up to 10 amino acid residues in length.

In another embodiment, the connecting peptide is up to 9, 8, 7, or 6 amino acid residues in length.

In a further embodiment, the connecting peptide is up to 5 amino acid residues in length and in a still further embodiment, the connecting peptide is up to 3 amino acid residues in length.

The B-chain may the full length insulin B chain; B(1-30), or a shortened B-chain. Thus it is well known in the art that up to 5 amino acid residues may be removed from either the N-terminal end or the B-terminal end or both of the human insulin B-chain without affecting the insulin activity adversely.

The insulin precursors or insulin analogue precursors will typically only be glycosylated in the connecting peptide. Furthermore, the connecting peptide will typically only contain one glycosylation site.

In a more specific embodiment the present invention is related to insulin precursors or insulin analogue precursors comprising the formula:

X₁-X₂-B(6-26)-X₃-X₄-A(1-21)

wherein

X₁ is a peptide sequence of 2-30 amino acids,

X₂ is a peptide sequence comprising one or more of the amino acid residues B1 to B5 from the N-terminal end of the human insulin B-chain and a cleavage site enabling cleavage from X₁,

X₃ is a peptide sequence of up to 14 amino acid residues in length comprising one or more of the amino acid residues B27 to B30 from the C-terminal end of the human insulin B-chain, and

X₄ is a cleavage site,

B(6-26) is the human insulin B-chain from amino acid residue number 6 to amino acid residue number 26,

and A(1-21) is the human insulin A chain,

wherein the sequence X₁-X₂ or X₃ or both contain at least one glycosylation site.

In one embodiment X₁ is 2-25, 2-20 or 2-15 amino acid residues in length. In another embodiment X₁ is 2-10 or 2-8 amino acid residues in length.

In another embodiment X₂ comprises the peptide sequence B(1-5), B(2-5), B(3-5), or B(4-5) of the human insulin B-chain. X₂ comprises preferably the peptide sequence B(1-5) and Lys or Arg as the cleavage site enabling cleavage from X₁.

X₁ will preferably comprise at least one negatively charged amino acid residue, such as Glu or Asp.

Examples of insulin precursors or insulin analogue precursors according to the present invention are such wherein the sequence X₃-X₄ is Ser-Asn-Thr-Thr-Lys (SEQ ID NO: 1), Ser-Ala-Asn-Asn-Thr-Lys (SEQ ID NO:4), Ser-Pro-Asn-Thr-Thr-Lys (SEQ ID NO:5), Ser-Ser-Asn-Thr-Thr-Lys (SEQ ID NO:6), Ser-Arg-Asn-Thr-Thr-Lys (SEQ ID NO:7) or Ala-Ala-Lys and the sequence X₁-X₂ is Glu-Glu-Gly-Asn-Thr-Thr-Glu-Pro-Lys (SEQ ID NO:3) or Glu-Glu-Gly-Glu-Pro-Lys (SEQ ID NO:2).

X₃ has to be in vitro cleavable from the C-terminal amino acid residue in the B-chain. If B29 is Lys as in human insulin cleavage can be accomplished by use of trypsin or trypsin like proteases which will cleave at the C-terminal of a Lys residue. Cleavage may also be accomplished by introducing a cleavage site such as Met cleavable by cyanogen bromide, Asn, Asn-Gly cleavable with hydroxylamine; Lys cleavable with Ahcromobacter lyticus protease or Armillaria mellea protease or a pair of basic amino acid residues (Lys or Arg) cleavable by kexin or yapsin from yeast or their homologues from other eukaryotic organisms. X₃ is cleavable from the A-chain at the cleavage site X₄. X₄ may be any convenient cleavage site, e.g. a Met cleavable by cyanogen bromide or an Asn, Asn-Gly cleavable with hydroxylamine; a single basic amino acid residue (Lys or Arg) cleavable by trypsin or trypsin like proteases; a lysine residue cleavable with Achromobacter lyticus protease or a pair of basic amino acid residues (Lys or Arg) cleavable by kexin or yapsin from yeast or their homologues from other eukaryotic organisms. The cleavage site X₄ enabling cleavage of X₃ from the A-chain is preferably a single basic amino acid residue Lys or Arg, preferably Lys.

Likewise, the N-terminal extension X₁ should be in vitro cleavable from the N-terminal end of the B-chain. This is accomplished by the sequence X₂ which comprises a cleavage site at its N-terminal end. X₂ may comprise any convenient cleavage site known in the art, e.g. a Met cleavable by cyanogen bromide or an Asn, Asn-Gly cleavable with hydroxylamine; a single basic amino acid residue (Lys or Arg) cleavable by trypsin or trypsin like proteases; a lysine residue cleavable with Ahcromobacter lyticus protease or a pair of basic amino acid residues (Lys or Arg) cleavable by kexin or yapsin from yeast or their homologues from other eukaryotic organisms.

Thus the insulin precursors may be X₁-X₂-B(6-29)-X₃-X₄-A(1-21); X₁-X₂-B(5-29)-X₃-X₄-A(1-21); X₁-X₂-B(4-29)-X₃-X₄-A(1-21); X₁-X₂-B(3-29)-X₃-X₄-A(1-21); X_{1-X}₂-B(2-29)-X₃-X₄-A(1-21); X₁-X₂-B(1-28)-Lys-X₃-X₄-A(1-21); X₁-X₂-B(1-27)-Lys-X₃-X₄-A(1-21); X₁-X₂-B(1-26)-Lys-X₃-X₄-A(1-21); X₁-X₂-B(2-28)-X₃-X₄-A(1-21); X₁-X₂-B(2-27)-X₃-X₄-A(1-21); X₁-X₂-B(2-26)-X₃-X₄-A(1-21); X₁-X₂-B(3-29)-X₃-X₄-A(1-21); X₁-X₂-B(3-28)-X₃-X₄-A(1-21); X₁-X₂-B(3-27)-X₃-X₄-A(1-21); X₁-X₂-B(3-26)-X₃-X₄-A(1-21); X₁-X₂-B(4-28)-X₃-X₄-A(1-21); X₁-X₂-B(4-27)-X₃-X₄-A(1-21); or X₁-X₂-B(4-26)-X₃-X₄-A(1-21) where X₁₋₄ have the above meanings.

Examples of combinations of C-peptides and N-terminal extensions according to the present invention are Ser-Asn-Thr-Thr-Lys (SEQ ID NO:1) (C-peptide) and Glu-Glu-Gly-Glu-Pro Lys (SEQ ID NO:2) (N-terminal extension), Ala-Ala-Lys (C-peptide) and Glu-Glu-Gly-Asn-Thr-Thr-Glu-Pro-Lys (SEQ ID NO:3) (N-terminal extension); Ser-Ala-Asn-Asn-Thr-Lys (SEQ ID NO:4) (C-peptide) and Glu-Glu-Gly-Glu-Pro Lys (SEQ ID NO:2) (N-terminal extension), Ser-Pro-Asn-Thr-Thr-Lys (SEQ ID NO:5) (C-peptide) and Glu-Glu-Gly-Glu-Pro Lys (SEQ ID NO:2) (N-terminal extension); Ser-Ser-Asn-Thr-Thr-Lys (SEQ ID NO:6) (C-peptide) and Glu-Glu-Gly-Glu-Pro Lys (SEQ ID NO:2) (N-terminal extension); or Ser-Arg-Asn-Thr-Thr-Lys (SEQ ID NO:7) (C-peptide) and Glu-Glu-Gly-Glu-Pro Lys (SEQ ID NO:2) (N-terminal extension).

The present invention is also related to polynucleotide sequences which code for the claimed insulin precursors or insulin analogue precursors. In a further aspect the present invention is related to vectors containing such polynucleotide sequences and host cell containing such polynucleotide sequences or vectors.

In another aspect, the invention relates to a process for producing the insulin precursors or insulin analogue precursors in a host cell, said method comprising (i) culturing a host cell comprising a polynucleotide sequence encoding the insulin precursors or insulin analogue precursors of the invention under suitable conditions for expression of said precursor; and (ii) isolating the precursor from the culture medium.

In still a further aspect, the invention relates to a process for producing insulin or insulin analogues in a host cell said method comprising (i) culturing a host cell comprising a polynucleotide sequence encoding an insulin precursor or insulin analogue precursors of the invention; (ii) isolating the precursor from the culture medium and (iii) converting the precursor into insulin or an insulin analogue by in vitro enzymatic conversion.

In one embodiment of the present invention the host cell is a yeast host cell and in a further embodiment the yeast host cell is selected from the genus Saccharomyces.

In a further embodiment the yeast host cell is selected from the species Saccharomyces cerevisiae.

### DETAILED DESCRIPTION

Abbreviations and Nomenclature.

By “connecting peptide” or “C-peptide” is meant the connection moiety “C” of the B-C-A polypeptide sequence of a single chain preproinsulin-like molecule. Specifically, in the natural insulin chain, the C-peptide connects position 30 of the B chain and position 1 of the A chain. A “mini C-peptide” or “connecting peptide” such as those described herein, connect B29 or B30 to A1, and differ in sequence and length from that of the natural C-peptide.

By “N-terminal extension” is meant a peptide chain which is attached at its C-terminal end to the N-terminal end of the B-chain or the shortened B-chain. The N-terminal extension is typically at its N-terminal end linked to a propeptide which is cleaved of from the N-terminal extension during secretion from the host cell.

By “insulin precursor” is meant a single-chain insulin precursor in which a desB25-desB30 chain is linked to the A chain of insulin via a connecting peptide. The single-chain insulin precursor will contain correctly positioned disulphide bridges (three) as in human insulin.

With “desB30” or “B(1-29)” is meant a natural insulin B chain lacking the B30 amino acid residue. With “B(6-26)” is meant the natural insulin B chain lacking the B(27-30) and the B(1-5) residues. “B(5-26)” means the natural insulin B chain lacking the B(1-4) and the B(27-30) residues etc. “B(1-27)” means the natural B chain lacking the B28, B29, and B30 amino acid residues, “B(1-28)” means the natural B chain lacking the B29 and B30 amino acid residues etc. “A(1-21)” means the natural insulin A chain,”

The “insulin precursor” can by one or more subsequent chemical and/or enzymatic processes be converted into human insulin.

By “insulin analogue precursor” is meant an insulin precursor molecule having one or more mutations, substitutions, deletions and or additions of the A and/or B amino acid chains relative to the human insulin molecule. The insulin analogues are preferably such wherein one or more of the naturally occurring amino acid residues, preferably one, two, or three of them, have been substituted by another codable amino acid residue. In one embodiment, the instant invention comprises analogue molecules having position 28 of the B chain altered relative to the natural human insulin molecule. In this embodiment, position 28 is modified from the natural Pro residue to one of Asp, Lys, or Ile. In a preferred embodiment, the natural Pro residue at position B28 is modified to an Asp residue. In another embodiment Lys at position B29 is modified to Pro; Also, Asn at position A21 may be modified to Ala, Gln, Glu, Gly, His, Ile, Leu, Met, Ser, Thr, Trp, Tyr or Val, in particular to Gly, Ala, Ser, or Thr and preferably to Gly. Furthermore, Asn at position B3 may be modified to Lys. Further examples of insulin precursor analogues are des(B30) human insulin, insulin analogues wherein Phe^{B1} has been deleted; insulin analogues wherein the A-chain and/or the B-chain have an N-terminal extension and insulin analogues wherein the A-chain and/or the B-chain have a C-terminal extension. Thus one or two Arg may be added to position B1.

The term “immediately N-terminal to” is meant to illustrate the situation where an amino acid residue or a peptide sequence is directly linked at its C-terminal end to the N-terminal end of another amino acid residue or amino acid sequence by means of a peptide bond.

By N-glycosylation site is meant a site generally known to allow substitution of the amide Nitrogen group of Asn with an oligosaccharide which in yeast consists of 14 monosaccharides: glucose₃mannose₉N-acetylglucosamine₂

“POT” is the Schizosaccharomyces pombe triose phosphate isomerase gene, and “TPI1” is the S. cerevisiae triose phosphate isomerase gene.

By a “leader” is meant an amino acid sequence consisting of a pre-peptide (the signal peptide) and a pro-peptide.

The term “signal peptide” is understood to mean a pre-peptide which is present as an N-terminal sequence on the precursor form of a protein. The function of the signal peptide is to allow the heterologous protein to facilitate translocation into the endoplasmic reticulum. The signal peptide is normally cleaved off in the course of this process. The signal peptide may be heterologous or homologous to the yeast organism producing the protein. A number of signal peptides which may be used with the DNA construct of the invention including yeast aspartic protease 3 (YAP3) signal peptide or any functional analog (Egel-Mitani et al. (1990) YEAST 6:127-137 and U.S. Pat. No. 5,726,038) and the α-factor signal of the MFα1 gene (Thorner (1981) in The Molecular Biology of the Yeast Saccharomyces cerevisiae, Strathern et al., eds., pp 143-180, Cold Spring Harbor Laboratory, N.Y. and U.S. Pat. No. 4,870,00.

The term “pro-peptide” means a polypeptide sequence whose function is to allow the expressed polypeptide to be directed from the endoplasmic reticulum to the Golgi apparatus and further to a secretory vesicle for secretion into the culture medium (i.e. exportation of the polypeptide across the cell wall or at least through the cellular membrane into the periplasmic space of the yeast cell). The pro-peptide may be the yeast α-factor pro-peptide, vide U.S. Pat. Nos. 4,546,082 and 4,870,008. Alternatively, the pro-peptide may be a synthetic pro-peptide, which is to say a pro-peptide not found in nature. Suitable synthetic pro-peptides are those disclosed in U.S. Pat. Nos. 5,395,922; 5,795,746; 5,162,498 and WO 98/32867. The pro-peptide will preferably contain an endopeptidase processing site at the C-terminal end, such as a Lys-Arg sequence or any functional analog thereof.

The polynucleotide sequence of the invention may be prepared synthetically by established standard methods, e.g. the phosphoamidite method described by Beaucage et al. (1981) Tetrahedron Letters 22:1859-1869, or the method described by Matthes et al. (1984) EMBO Journal 3:801-805. According to the phosphoamidite method, oligonucleotides are synthesized, for example, in an automatic DNA synthesizer, purified, duplexed and ligated to form the synthetic DNA construct. A currently preferred way of preparing the DNA construct is by polymerase chain reaction (PCR).

The polynucleotide sequence of the invention may also be of mixed genomic, cDNA, and synthetic origin. For example, a genomic or cDNA sequence encoding a leader peptide may be joined to a genomic or cDNA sequence encoding the A and B chains, after which the DNA sequence may be modified at a site by inserting synthetic oligonucleotides encoding the desired amino acid sequence for homologous recombination in accordance with well-known procedures or preferably generating the desired sequence by PCR using suitable oligonucleotides.

The invention encompasses a vector which is capable of replicating in the selected microorganism or host cell and which carries a polynucleotide sequence encoding the insulin precursors or insulin analogue precursors of the invention. The recombinant vector may be an autonomously replicating vector, i.e., a vector which exists as an extra-chromosomal entity, the replication of which is independent of chromosomal replication e.g., a plasmid, an extra-chromosomal element, a mini-chromosome, or an artificial chromosome. The vector may contain any means for assuring self-replication. Alternatively the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell, or a transposon may be used. The vector may be linear or closed circular plasmids and will preferably contain an element(s) that permits stable integration of the vector into the host cell's genome or autonomous replication of the vector in the cell independent of the genome.

In a preferred embodiment, the recombinant expression vector is capable of replicating in yeast Examples of sequences which enable the vector to replicate in yeast are the yeast plasmid 2 μm replication genes REP 1-3 and origin of replication.

The vectors of the present invention preferably contain one or more selectable markers which permit easy selection of transformed cells. A selectable marker is a gene the product of which provides for biocide or viral resistance, resistance to heavy metals, prototrophy to auxotrophs, and the like. Examples of bacterial selectable markers are the dal genes from Bacillus subtilis or Bacillus licheniformis, or markers which confer antibiotic resistance such as ampicillin, kanamycin, chloramphenicol or tetracycline resistance. Selectable markers for use in a filamentous fungal host cell include amdS (acetamidase), argB (ornithine carbamoyltransferase), pyrG (orotidine-5′-phosphate de-carboxylase) and trpC (anthranilate synthase. Suitable markers for yeast host cells are ADE2, HIS3, LEU2, LYS2, MET3, TRP1, and URA3. A preferred selectable marker for yeast is the Schizosaccharomyces pompe TPI gene (Russell (1985) Gene 40:125-130).

In the vector, the polynucleotide sequence is operably connected to a suitable promoter sequence. The promoter may be any nucleic acid sequence which shows transcriptional activity in the host cell of choice including mutant, truncated, and hybrid promoters, and may be obtained from genes encoding extra-cellular or intra-cellular polypeptides either homologous or heterologous to the host cell.

Examples of suitable promoters for directing the transcription in a bacterial host cell, are the promoters obtained from the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), and Bacillus licheniformis penicillinase gene (penP). Examples of suitable promoters for directing the transcription in a filamentous fungal host cell are promoters obtained from the genes for Aspergillus oryzae TAKA amylase, Rhizomucor miehei aspartic proteinase, Aspergillus niger neutral alpha-amylase, and Aspergillus niger acid stable alpha-amylase. In a yeast host, useful promoters are the Saccharomyces cerevisiae Ma1, TPI, ADH or PGK promoters.

The polynucleotide construct of the invention will also typically be operably connected to a suitable terminator. In yeast a suitable terminator is the TPI terminator (Alber et al. (1982) J. Mol. Appl. Genet. 1:419-434).

The procedures used to ligate the polynucleotide sequence of the invention, the promoter and the terminator, respectively, and to insert them into suitable yeast vectors containing the information necessary for yeast replication, are well known to persons skilled in the art. It will be understood that the vector may be constructed either by first preparing a DNA construct containing the entire DNA sequence encoding the insulin precursors or insulin analogue precursors of the invention, and subsequently inserting this fragment into a suitable expression vector, or by sequentially inserting DNA fragments containing genetic information for the individual elements (such as the signal, pro-peptide, mini C-peptide, A and B chains) followed by ligation.

The present invention also relates to recombinant host cells, comprising a polynucleotide sequence encoding the insulin precursors or the insulin analogue precursors of the invention. A vector comprising such polynucleotide sequence is introduced into the host cell so that the vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the polypeptide and its source. The host cell may be a unicellular microorganism, e.g., a prokaryote, or a non-unicellular microorganism, e.g., a eukaryote. Useful unicellular cells are bacterial cells such as gram positive bacteria including, but not limited to, a Bacillus cell, Streptomyces cell, or gram negative bacteria such as E. coli and Pseudomonas sp. Eukaryote cells may be mammalian, insect, plant, or fungal cells. In a preferred embodiment, the host cell is a yeast cell. The yeast organism used in the process of the invention may be any suitable yeast organism which, on cultivation, produces large amounts of the insulin precursor and insulin analogue precursors of the invention. Examples of suitable yeast organisms are strains selected from the yeast species Saccharomyces cerevisiae, Saccharomyces kluyveri, Schizosaccharomyces pombe, Sacchoromyces uvarum, Kluyveromyces lactis, Hansenula polymorpha, Pichia pastoris, Pichia methanolica, Pichia kluyveri, Yarrowia lipolytica, Candida sp., Candida utilis, Candida cacaoi, Geotrichum sp., and Geotrichum fermentans.

The transformation of the yeast cells may for instance be effected by protoplast formation followed by transformation in a manner known per se. The medium used to cultivate the cells may be any conventional medium suitable for growing yeast organisms. The secreted insulin precursor or insulin analogue precursors of the invention, a significant proportion of which will be present in the medium in correctly processed form, may be recovered from the medium by conventional procedures including separating the yeast cells from the medium by centrifugation, filtration or catching the insulin precursor or insulin analogue precursor by an ion exchange matrix or by a reverse phase absorption matrix, precipitating the proteinaceous components of the supernatant or filtrate by means of a salt, e.g. ammonium sulphate, followed by purification by a variety of chromatographic procedures, e.g. ion exchange chromatography, affinity chromatography, or the like.

After secretion to the culture medium the insulin precursors may conveniently be separated from the culture broth by affinity chromatography on a column which is capable of binding the sugar molecule(s) attached to the insulin precursor molecule.

After recovery, the insulin precursor or insulin analogue precursors of the invention will be subjected to various in vitro procedures to remove the N-terminal extension sequence and the C-peptide to give insulin or the desired insulin analogue as described above.

Cleavage of the connecting peptide from the B chain is preferably enabled by cleavage at the natural Lys^{B29} amino acid residue in the B chain giving rise to a desB30 insulin precursor or desB30 insulin analogue precursor. If the insulin precursor is to be converted into human insulin, the B30Thr amino acid residue can be added by well known in vitro, enzymatic procedures such methods include enzymatic conversion by means of trypsin or an Achromobacter lyticus protease in the presence of an L-threonine ester followed by conversion of the threonine ester of the insulin into insulin by basic or acid hydrolysis as described in U.S. Pat. Nos. 4,343,898 or 4,916,212. The desB30 insulin may also be converted into an acylated insulin as disclosed in U.S. Pat. No. 5,750,497 and U.S. Pat. No. 5,905,140 the disclosures of which are incorporated by reference hereinto.

The present invention is described in further detail in the following examples which are not in any way intended to limit the scope of the invention as claimed. The attached Figures are meant to be considered as integral parts of the specification and description of the invention. All references cited are herein specifically incorporated by reference for all that is described therein.

### EXAMPLES

General Procedures

All expressions plasmids are of the C-POT type, similar to those described in EP 171,142, which are characterized by containing the Schizosaccharomyces pombe triose phosphate isomerase gene (POT) for the purpose of plasmid selection and stabilization in S. cerevisiae. The plasmids also contain the S. cerevisiae triose phosphate isomerase promoter and terminator. These sequences are similar to the corresponding sequences in plasmid pKFN1003 (described in WO 90/100075) as are all sequences except the sequence of the EcoRI-Xbal fragment encoding the fusion protein of the propeptide and the insulin precursor or insulin precursor analogue in question.

Yeast transformants were prepared by transformation of the host strain S. cerevisiae strain MT663 (MATa/MATα pep4-3/pep4-3 HIS4/his4 tpi::LEU2/tpi::LEU2 Cir⁺). The yeast strain MT663 was deposited in the Deutsche Sammlung von Mikroorganismen und Zellkulturen in connection with filing WO 92/11378 and was given the deposit number DSM 6278.

MT663 was grown on YPGaL (1% Bacto yeast extract, 2% Bacto peptone, 2% galactose, 1% lactate) to an O.D. at 600 nm of 0.6. 100 ml of culture was harvested by centrifugation, washed with 10 ml of water, recentrifuged and resuspended in 10 ml of a solution containing 1.2 M sorbitol, 25 mM Na₂EDTA pH=8.0 and 6.7 mg/ml dithiotreitol. The suspension was incubated at 30° C. for 15 minutes, centrifuged and the cells resuspended in 10 ml of a solution containing 1.2 M sorbitol, 10 mM Na₂EDTA, 0.1 M sodium citrate, pH 0 5.8, and 2 mg Novozym®234. The suspension was incubated at 30° C. for 30 minutes, the cells collected by centrifugation, washed in 10 ml of 1.2 M sorbitol and 10 ml of CAS (1.2 M sorbitol, 10 mM CaCl₂, 10 mM Tris HCl (Tris=Tris(hydroxymethyl)aminomethane) pH=7.5) and resuspended in 2 ml of CAS. For transformation, 1 ml of CAS-suspended cells was mixed with approx. 0.1 mg of plasmid DNA and left at room temperature for 15 minutes. 1 ml of (20% polyethylene glycol 4000, 10 mM CaCl₂, 10 mM Tris HCl, pH=7.5) was added and the mixture left for a further 30 minutes at room temperature. The mixture was centrifuged and the pellet resuspended in 0.1 ml of SOS (1.2 M sorbitol, 33% v/v YPD, 6.7 mM CaCl₂) and incubated at 30° C. for 2 hours. The suspension was then centrifuged and the pellet resuspended in 0.5 ml of 1.2 M sorbitol. Then, 6 ml of top agar (the SC medium of Sherman et al. (1982) Methods in Yeast Genetics, Cold Spring Harbor Laboratory) containing 1.2 M sorbitol plus 2.5% plus 2.5% agar) at 52° C. was added and the suspension poured on top of plates containing the same agar-solidified, sorbitol containing medium.

S. cerevisiae strain MT663 was transformed with expression plasmids comprising DNA encoding the insulin precursor in question and was grown in YPD medium (2% Bacto yeast extract, 1% Bacto peptone, 6% glucose) for 72 h at 30° C. Quantitation of the insulin-precursor yield in the culture supernatants was performed by reverse-phase HPLC analysis with human insulin as an external standard (Snel & Damgaard (1988) Pro-insulin heterogenity in pigs. Horm. Metabol. Res. 20:476-488) after conversion to desB30 insulin after treatment with ALP enzyme.

### Example 1

Expression of Insulin Analogue Precursors Wherein the B(1-29) Chain is Connected to the A(1-21) Chain via a Connection Peptide AAK, SEQ ID NO:1, SEQ ID NO:4, SEQ ID NO:5, SEQ ID NO:6 or SEQ ID NO:7.

Expression was conducted in yeast as described above. Strains were grown in 500 ml shake flasks approximately 200 ml YPD medium. The precursors have an N-terminal extension EEGNTTEPK (SEQ ID NO:3) or EEGEPK (SEQ ID NO:2). All insulin precursors according to the invention were furnished with the YAP3 signal and a synthetic leader sequence named TA39 as disclosed in WO 02/00191 or WO 02/00190 and were expressed as a fusion protein e.g.: “YAP3-signal-TA39-leader-N-terminally-extended-insulin-precursor”. The signal-leader sequence is cleaved off during secreting. Expression results of the N-terminally extended insulin precursor in question measured by HPLC are shown in Table 1 as a percent of the control which is the insulin precursor B(1-29)-Ala-Ala-Lys-A(1-21).

TABLE 1

| | | Yield of N-terminally |
|---|---|---|
| N-terminal | | extended insulin precursor |
| extension | C-peptide | in mg/l in % of control |
| No | AAK | 100 |
| | | (control) |
| EEGEPK | AAK | 273 |
| (SEQ ID NO:2) | | |
| EEGNTTEPK | AAK | 300 |
| (SEQ ID NO:3) | | |
| EEGEPK | SNTTK | 602 |
| (SEQ ID NO:2) | (SEQ ID NO:1) | |
| EEGNTTEPK | SNTTK | 446 |
| (SEQ ID NO:3) | (SEQ ID NO:1) | |
| EEGEPK | SANNTK | 488 |
| (SEQ ID NO:2) | (SEQ ID NO:4) | |
| EEGEPK | SPNTTK | 343 |
| (SEQ ID NO:2) | (SEQ ID NO:5) | |
| EEGEPK | SSNTTK | 654 |
| (SEQ ID NO:2) | (SEQ ID NO:6) | |
| EEGEPK | SRNTTK | 368 |
| (SEQ ID NO:2) | (SEQ ID NO:7) | |

What is claimed is:

1. An insulin precursor or insulin analogue precursor comprising: (i) a connecting peptide being cleavable from the A- and B-chains of insulin and (ii) an N-terminal extension comprising a cleavage site immediately N-terminal to the B-chain, wherein the connecting peptide is 1-10 amino acid residues in length and wherein the connecting peptide, the N-terminal extension or both contain at least one glycosylation site.

2. An insulin precursor or insulin analogue precursor according to claim 1, wherein the connecting peptide is 1-6 amino acid residues in length.

3. An insulin precursor or insulin analogue precursor according to claim 1, wherein the connecting peptide is 1-3 amino acid residues in length.

4. An insulin precursor or insulin analogue precursor according to claim 1, wherein only the connecting peptide contains at least one glycosylation site.

5. An insulin precursor or insulin analogue precursor according to claim 4, wherein the connecting peptide contains one glycosylation site.

6. An insulin precursor or insulin analogue precursor according to claim 1, wherein the cleavage site enabling cleavage of the peptide bond between the A-chain and the connecting peptide is Lys or Arg.

7. An insulin precursor or insulin analogue precursor according to claim 1, wherein the N-terminal extension is 1-25 amino acid residues in length.

8. An insulin precursor or insulin analogue precursor according to claim 7, wherein the N-terminal extension is 1-20 amino acid residues in length.

9. An insulin precursor or insulin analogue precursor according to claim 7, wherein the N-terminal extension 1-5 amino acid residues in length.

10. An insulin precursor or insulin analogue precursor according to claim 1, wherein the cleavage site in the N-terminal extension is selected from the group consisting of Met, Lys-Arg, Lys-Lys, Arg-Lys, Arg-Arg, Lys, and Arg.

11. An insulin precursor or insulin analogue precursor according to claim 1, wherein the connecting peptide is selected from the group consisting of:
Ser-Asn-Thr-Thr-Lys (SEQ ID NO:1),
Ser-Ala-Asn-Asn-Thr-Lys (SEQ ID NO:4),
Ser-Pro-Asn-Thr-Thr-Lys (SEQ ID NO:5),
Ser-Ser-Asn-Thr-Thr-Lys (SEQ ID NO:6),
Ser-Arg-Asn-Thr-Thr-Lys (SEQ ID NO:7) and
Ala-Ala-Lys.

12. An insulin precursor or insulin analogue precursor according to claim 1, wherein the N-terminal extension is Glu-Glu-Gly-Asn-Thr-Thr-Glu-Pro-Lys (SEQ ID NO:3) or Glu-Glu-Gly-Glu-Pro-Lys (SEQ ID NO: 2).

13. An insulin precursor or an insulin analogue precursor according to claim 1 comprising the formula:
X₁-X₂-B(6-26)-X₃-X₄-A(1-21)
wherein
X₁ is a peptide sequence of 2-30 amino acids,
X₂ is a peptide sequence comprising one or more of the amino acid residues B₁ to B5 from the N-terminal end of the human insulin B-chain and a cleavage site enabling cleavage from X₁,
X₃ is a peptide sequence of up to 14 amino acid residues in length comprising one or more of the amino acid residues B27 to B30 from the C-terminal end of the human insulin B-chain, and
X₄ is a cleavage site,
B(6-26) is the human insulin B-chain from amino acid residue number 6 to amino acid residue number 26,
and A(1-21) is the human insulin A chain,
wherein one or more of the sequences X₁-X₂ and X₃ contain at least one glycosylation site.

14. An insulin precursor or insulin analogue precursor of claim 13, wherein X₁ is 2-25 amino acid residues in length.

15. An insulin precursor or insulin analogue precursor of claim 13, wherein X₁ is 2-10 amino acid residues in length.

16. An insulin precursor or insulin analogue precursor according to claim 13, wherein X₂ comprises a cleavage site consisting of Lys or Arg.

17. An insulin precursor or insulin analogue precursor according to claim 13, wherein X₂ comprises the peptide sequence B(1-5), B(2-5), B(3-5), or B(4-5) of the human insulin B-chain.

18. An insulin precursor or insulin analogue precursor according to claim 13, wherein X₂ comprises (i) the peptide sequence B(1-5); and (ii) Lys or Arg as the cleavage site enabling cleavage from X₁.

19. An insulin precursor or insulin analogue precursor according to claim 13, wherein X₃ comprises the peptide sequence B(27-29) or B(27-28) of the human insulin B-chain.

20. An insulin precursor or insulin analogue precursor according to claim 13, wherein X₄ is Lys or Arg.

21. An insulin precursor or insulin analogue precursor according to claim 13, wherein the sequence X₃-X₄ is selected from the group consisting of:
Ser-Asn-Thr-Thr-Lys (SEQ ID NO: 1),
Ser-Ala-Asn-Asn-Thr-Lys (SEQ ID NO:4),
Ser-Pro-Asn-Thr-Thr-Lys (SEQ ID NO:5),
Ser-Ser-Asn-Thr-Thr-Lys (SEQ ID NO:6),
Ser-Arg-Asn-Thr-Thr-Lys (SEQ ID NO:7) and
Ala-Ala-Lys.

22. An insulin precursor or insulin analogue precursor according to claim 13, wherein the sequence X₁-X₂ is Glu-Glu-Gly-Asn-Thr-Thr-Glu-Pro-Lys (SEQ ID NO:3) or Glu-Glu-Gly-Glu-Pro-Lys (SEQ ID NO:2).

23. An insulin precursor or insulin analogue precursor according to claim 13, wherein
(i) the sequence X₃-X₄ is selected from the group consisting of:
Ser-Asn-Thr-Thr-Lys (SEQ ID NO: 1),
Ser-Ala-Asn-Asn-Thr-Lys (SEQ ID NO:4),
Ser-Pro-Asn-Thr-Thr-Lys (SEQ ID NO:5),
Ser-Ser-Asn-Thr-Thr-Lys (SEQ ID NO:6),
Ser-Arg-Asn-Thr-Thr-Lys (SEQ ID NO:7) and
Ala-Ala-Lys; and
(ii) the sequence X₁-X₂ is Glu-Glu-Gly-Asn-Thr-Thr-Glu-Pro-Lys (SEQ ID NO:3) or Glu-Glu-Gly-Glu-Pro-Lys (SEQ ID NO:2).

24. A polynucleotide sequence encoding an insulin precursor or insulin analogue precursor according to claim 1.

25. An expression vector comprising a polynucleotide sequence according to claim 24.

26. A host cell transformed with a vector of claim 25.

27. A process for making an insulin precursor or an insulin analogue precursor said method comprising (i) culturing a host cell comprising a polynucleotide sequence encoding an insulin precursor or an insulin analogue precursor according to claim 1 under suitable culture conditions for expression of said precursor; and (ii) isolating the expressed precursor.

28. A process according to claim 27, wherein the host cell is yeast.

29. A process for making insulin or an insulin analogue, said method comprising (i) culturing a host cell comprising a polynucleotide sequence encoding an insulin precursor or an insulin analogue precursor according to claim 1 under suitable culture conditions for expression of said precursor; (ii) isolating the precursor from the culture medium and (iii) converting the precursor into insulin or an insulin analogue by in vitro chemical or enzymatic conversion.

30. A process according to claim 29, wherein the host cell is yeast.


SEQUENCE LISTING (7 sequences)

SEQ ID NO: 1 - protein, 5 aa, Artificial Sequence
  Synthetic
SNTTK

SEQ ID NO: 2 - protein, 6 aa, Artificial Sequence
  Synthetic
EEGEPK

SEQ ID NO: 3 - protein, 9 aa, Artificial Sequence
  Synthetic
EEGNTTEPK

SEQ ID NO: 4 - protein, 6 aa, Artificial Sequence
  Synthetic
SANNTK

SEQ ID NO: 5 - protein, 6 aa, Artificial Sequence
  Synthetic
SPNTTK

SEQ ID NO: 6 - protein, 6 aa, Artificial Sequence
  Synthetic
SSNTTK

SEQ ID NO: 7 - protein, 6 aa, Artificial Sequence
  Synthetic
SRNTTK